(19) United States
(12) Patent Application Publication
(10) Pub. No.: US 2026/0041377 A1
(43) Pub. Date: Feb. 12, 2026

(54) METHOD AND APPARATUS FOR NON-INVASIVELY COMPUTING CARDIO-VASCULATURE PARAMETERS USING MORPHOLOGY OF UNCORRELATED PRESSURE WAVE SIGNAL

(71) Applicant: ECOLE POLYTECHNIQUE FEDERALE DE LAUSANNE (EPFL), Lausanne (CH)

(72) Inventors: Vasiliki BIKIA, Lausanne (CH); Nikolaos STERGIOPULOS, Preverenges (CH)

(73) Assignee: ECOLE POLYTECHNIQUE FEDERALE DE LAUSANNE (EPFL), Lausanne (CH)

(21) Appl. No.: 19/102,164

(22) PCT Filed: Aug. 8, 2023
(86) PCT No.: PCT/IB2023/057976

§ 371 Date: Feb. 7, 2025

Related U.S. Application Data
(60) Provisional application No. 63/370,841, filed on Aug. 9, 2022.

(51) Int. Cl. A61B 5/00; A61B 5/022; A61B 5/024

(52) U.S. Cl. CPC A61B 5/7264; A61B 5/02233; A61B 5/02438; A61B 5/6833; A61B 2560/0228

(57) ABSTRACT

Methods are provided for estimating key reliably and accurately predicting cardiac output using a limited set of non-invasively monitored physiologic inputs, and a calibrated one-dimensional arterial tree model, a database of synthetic data generated from such a model, and an artificial intelligence module. Systems for estimating CO based on non-invasively measured physiologic inputs also are provided that may be implemented without the need for extensive real-time computing resources.

### CROSS-REFERENCE TO RELATED APPLICATIONS

This application claims priority to U.S. Provisional Patent Application No. 63/370,841, filed Aug. 9, 2022, the entire contents of which are incorporated herein by reference.

### FIELD OF THE INVENTION

The present invention relates to methods and apparatus for using a non-invasively monitored uncorrelated pressure signal from a patient to compute cardiac output and other cardio-vascular parameters, thereby providing safe, effective, reliable and low cost monitoring of patient health.

### BACKGROUND

International Application Publication No. WO 2021/033097, which is incorporated herein by reference, describes methods and apparatus for using non-invasively measured physiologic data, such as blood pressure and pulse wave propagation information, to predict in real-time compute noninvasively unobservable cardiovascular parameters, such as cardiac output, central systolic blood pressure and others. Such values generally are determinable only by way of invasive measurements. In that published application, it is described that a one-dimensional arterial tree may be trained on a limited dataset for a representative patient population, and that trained model then used to generate synthetic data for a larger virtual patient population using an artificial intelligence module. The resulting database for the expanded virtual patient population then may be used to determine key cardiovascular parameters in real-time based only a limited set of non-invasively measured patient data.

Although the methods and apparatus described WO 2021/033097 provide a quick and cost-effective system to obtain estimates of critical cardio-vascular information without invasive measurement, that system still required noninvasive measurement of multiple physiologic parameters, as well as ECG signals and the acoustic detection of heart sounds. Nonetheless, the systems and methods described therein demonstrated the feasibility of using non-invasively measured data to provide accurate real-time estimates of cardiovascular parameters critical to monitoring and assessing patient health.

In view of the foregoing, there exists a need for methods and apparatus for estimating key cardiovascular parameters using a simplified set of non-invasively monitored physiologic inputs.

It further would be desirable to provide methods and apparatus for predicting key cardiovascular parameters in real time using a limited set of non-invasively monitored physiologic inputs that employs robust software configured to be run without needing extensive computing resources.

### SUMMARY OF THE INVENTION

The present invention is directed methods and apparatus for estimating key cardiovascular parameters using a simplified set of non-invasively monitored physiologic inputs. In accordance with one aspect of the invention, robust algorithms are provided that are suitable for reliably and accurately predicting cardiac output, CO, using a limited set of non-invasively monitored physiologic inputs, and a calibrated one-dimensional arterial tree model, a database of synthetic data generated from such a model, and an artificial intelligence module. In this way, a system for estimating CO based on non-invasively measured physiologic inputs may be implemented without the need for extensive real-time computing resources.

Based on experience gained in developing the system and methods described in the above WO publication, it was inventors' insight that a correlation between the diastolic part of the aortic pressure (observable only by invasive methods) and carotid or temporal pressure (which may be non-invasively measured) could be established. As described herein, constant T, corresponding to the time for decay of diastolic carotid pressure or diastolic temporal pressure, closely correlates to time for decay of aortic pressure in all age groups from 30 to greater than 50 years old. Based on that correlation, it then is possible to compute an estimate cardiac output using simple formulas that relate CO to measurements of brachial systolic and diastolic blood pressure, heart rate (HR), and/or pulse wave velocity (PWV) data. Other features of the inventive system and methods will be apparent with reference to the following description and figures.

### BRIEF DESCRIPTION OF THE DRAWINGS

FIG. 1 is a schematic diagram illustrating derivation of diastolic pressure decay time constant T.

FIGS. 2A and 2B are, respectively, a plot of comparing aortic t and carotid t and aortic t and temporal t computed using synthetic data.

FIG. 3 is a plot showing correlation between t and the product of the heart beat duration times the mean arterial pressure divided by the aortic pulse pressure.

FIG. 4 is a flowchart of a first method of estimating a value of non-invasive cardiac output of the present invention.

FIGS. 5A and 5B are, respectively, a scatterplot and Bland-Altman plot showing correlation between actual and estimated values for total arterial compliance, while FIGS. 5C and 5D are, respectively, a scatterplot and Bland-Altman plot showing correlation between actual and estimated values of aortic pressure pulse computed according to the first method of the present invention.

FIGS. 6A and 6B are, respectively, a scatterplot and Bland-Altman plot showing correlation between actual and estimated values for cardiac output according to the first method of the present invention.

FIGS. 7A and 7B are, respectively, a scatterplot and Bland-Altman plot comparing predicted aortic mean pressure to true aortic mean pressure.

FIG. 8 is a flowchart of a second method of estimating a value of non-invasive cardiac output of the present invention.

FIGS. 9A and 9B are, respectively, a scatterplot and Bland-Altman plot showing a consistent bias in the preliminary results of actual and estimated values for cardiac output according to the second method of the present invention;

FIGS. 10A and 10B are, respectively, a scatterplot and Bland-Altman plot showing corrected values of actual and estimated values for cardiac output according to the second method of the present invention;

FIG. 11 is a schematic representation of a non-invasive system of measuring cardiac output constructed in accordance with the principles of the present invention.

### DETAILED DESCRIPTION OF THE INVENTION

The present invention is directed to methods for estimating cardiac output (“CO”) from uncalibrated non-invasively measured physiologic inputs of a patient and systems implementing such methods. The methods are based the inventors' insight that for large patient populations, the curve for exponential decay of the diastolic aortic pressure, which must be measured invasively, may be correlated to curve for exponential decay of diastolic pressure measured non-invasively at the carotid and temporal arteries. In particular, a one-dimensional model for the aortic tree, calibrated with data from a large patient population, as described in the above-mentioned WO application, was used to generate a database containing additional synthetic data that related aortic central pressure to carotid and temporal artery pressures. Empirical models then were derived for diastolic pressure decay constant t, computed as total peripheral resistance R multiplied by total arterial compliance C. Curve fitting analyses established that the value of diastolic pressure decay constant t for aortic pressure closely correlates to diastolic pressure decay constants for both carotid and temporal arteries, thus yielding a parameter that may be used to simplify CO estimation.

Next, two different formulations for cardiac output were developed, such that the diastolic pressure decay constant t could be used, together with synthetic data generated from the one-dimensional model and non-invasively measured values, to estimate CO. In a first model, CO is computed as a function of total arterial compliance C multiplied by the aortic pulse pressure aPP, divided by the heart beat interval T. In this first model, total arterial compliance is obtained from the synthetic data database using as inputs non-invasively measured blood pressure measurements for systolic and diastolic blood pressure, and carotid-femoral pulse wave velocity or/and carotid-radial pulse wave velocity values, as described in the above-mentioned WO application and summarized in V. Bikia, G. Rovas, S. Pagoulatou, and N. Stergiopulos, “Determination of Aortic Characteristic Impedance and Total Arterial Compliance From Regional Pulse Wave Velocities Using Machine Learning: An in-silico Study,” Front. Bioeng. Biotechnol., vol. 9, p. 649866, May 2021, doi: 10.3389/fbioe.2021.649866.

In a second model, CO is computed as a function of aortic mean pressure divided by the arterial peripheral resistance. In this case, arterial peripheral resistance is computed from T, which in turn is calculated from the carotid pressure waveform and the total arterial compliance predicted from systolic and diastolic blood pressure, carotid-femoral pulse wave velocity or carotid-radial pulse wave velocity values and heart rate values, as described for the first model above. In this second model, the uncalibrated carotid pressure waveform is calibrated employing the assumptions that (i) mean arterial pressure is computed as ⅓ times systolic blood pressure plus ⅔ diastolic blood pressure and (ii) diastolic blood pressure remains constant across all major arteries. In this manner, CO may be readily estimated using a one-dimensional arterial tree model as described above, or a calibrated arterial tree model may be used parametrically to generate a synthetic database for all physiologically relevant values, and then an artificial intelligence model employed with that databased and the noninvasively measured physiologic values to estimate CO.

The instant disclosure describes the assumptions and bases leading to derivation of the novel correlation for the arterial pressure time decay constant, validation of those assumptions using actual patient data, extended using additional synthetic data generated for physiologically relevant cases, and then alternative models of using the validated arterial pressure decay constant to compute CO for a patient using only noninvasively measured physiologic inputs and the arterial tress model or database generated therefrom and an AI module. Finally, a system that implements the above models to provide an exemplary patient monitor for use in estimating CO for use in a health care environment is described.

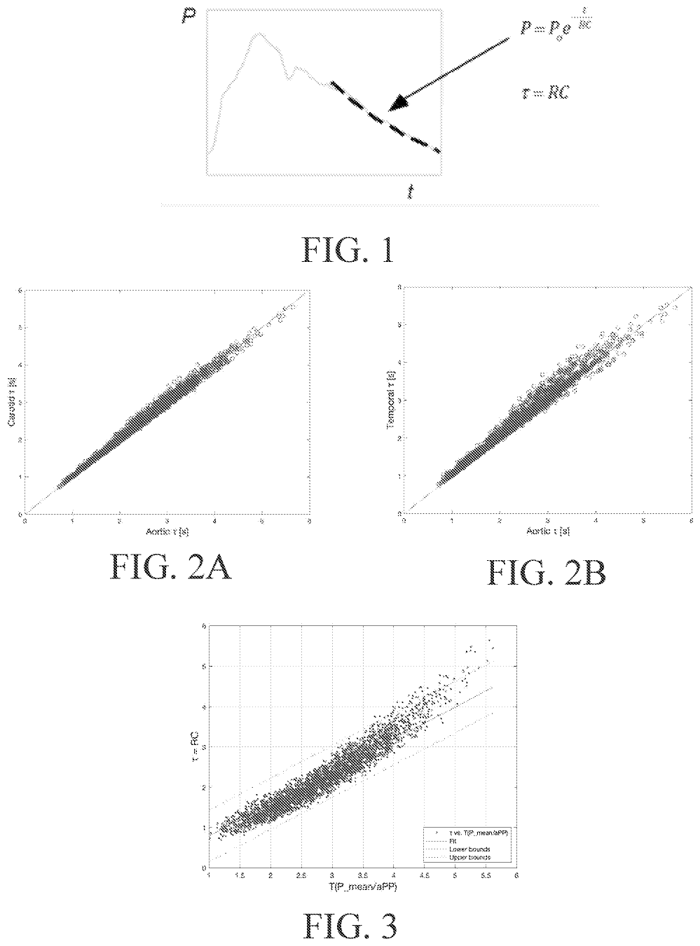

Referring to FIG. 1, as described in the article by N. Westerhof, N. Stergiopulos, and M. I. M. Noble, Snapshots of Hemodynamics: An Aid for Clinical Research and Graduate Education, 2nd ed. Springer, 2010, the exponential arterial pressure decay in diastole may be characterized by a time constant, T. When blood flow is zero, as in diastole, the decrease of aortic pressure, is characterized by the decay time t, equals the product of R and C, where R is total peripheral resistance and C is the arterial compliance in accordance with Equation 1:

$$τ = R \times C \qquad ( Equation l )$$

In this case, τ may be calculated from the blood pressure waveform, P, depicted in FIG. 1, by fitting a mono-exponential decay function to the diastolic part of the curve (FIG. 1, dashed line).

It is known that the contour of the arterial pressure pulse varies dramatically during its transmission down the arterial tree, and that pressure decay during diastole typically is not a monotonic function of time, as described in E. J. Kroeker and E. H. Wood, “Beat-to-Beat Alterations in Relationship of Simultaneously Recorded Central and Peripheral Arterial Pressure Pulses During Valsalva Maneuver and Prolonged Expiration in Man,” Journal of Applied Physiology, vol. 8, no. 5, pp. 483-494, March 1956, doi: 10.1152/jappl.1956.8.5.483 and E. J. Kroeker and E. H. Wood, “Comparison of Simultaneously Recorded Central and Peripheral Arterial Pressure Pulses During Rest, Exercise and Tilted Position in Man,” Circulation Research, vol. 3, no. 6, pp. 623-632, November 1955, doi: 10.1161/01.RES.3.6.623. However, as reported in the above-mentioned article by Westerhof et al., a relatively smooth diastolic pressure decay has been observed for aortic pressure pulses and also has been shown to be of pathophysiological relevance. For example, J. Hashimoto and S. Ito, “Central diastolic pressure decay mediates the relationship between aortic stiffness and myocardial viability: potential implications for aortosclerosis-induced myocardial ischemia,” Journal of Hypertension, vol. 35, no. 10, pp. 2034-243 October 2017, doi: 10.1097/HJH.0000000000001436, highlights the importance of aortic diastolic pressure decay caused by arterial stiffening in the pathophysiology of ischemic myocardial disease.

Similarly, J. Izzo, M. Anwar, S. Elsayed, P. Osmond, and B. Gavish, “Implications Of Diastolic Pressure-Decay Differences In The Radial And Carotid Arteries:” Journal of Hypertension, vol. 37, p. e317, July 2019, doi: 10.1097/01.hjh.0000573952.91074.62, compared the diastolic decay between carotid and radial arterial pressure waves and found that carotid t is higher than radial t and the two variables are not correlated, although both are affected by arterial stiffening. Despite this apparent non-correlation, the carotid blood pressure wave often is used as a surrogate of central (aortic) blood pressure. Heretofore, due to the intrinsic difficulty in acquiring concurrent invasive data at the aorta and the carotid artery or the temporal artery, it was unknown whether aortic diastolic decay, and thus t, shares a common pattern with the carotid diastolic decay.

Notwithstanding the foregoing, the present inventors postulated that a positive correlation could be established between the aortic and carotid diastolic decay. Specifically, the calibrated one-dimensional arterial tree model described in the above-incorporated WO application was employed to generate an extended database of synthetic data for physiologically relevant values. This data then was used to compare t derived from carotid and temporal pressure with t derived from the aortic pressure. Further details of the mathematical model of the cardiovascular system are described in P. Reymond, F. Merenda, F. Perren, D. Rüfenacht, and N. Stergiopulos, “Validation of a one-dimensional model of the systemic arterial tree,” Am. J. Physiol. Heart Circ. Physiol., vol. 297, no. 1, pp. H208-222, July 2009, doi: 10.1152/ajpheart.00037.2009 and P. Reymond, Y. Bohraus, F. Perren, F. Lazeyras, and N. Stergiopulos, “Validation of a patient-specific one-dimensional model of the systemic arterial tree,” Am. J. Physiol. Heart Circ. Physiol., vol. 301, no. 3, pp. H1173-1182 September 2011, doi: 10.1152/ajpheart.00821.2010.

FIGS. 2A and 2B are plots showing agreement between the aortic t and the carotid/temporal τ values using the above methodology, thus validating the inventors' hypothesis. Table 1 below provides metrics for those comparisons, where MAE corresponds to the mean absolute error, in seconds.

TABLE 1

| | Correlation | MAE [s] |
|---|---|---|
| Aortic τ vs Carotid τ | 1 | 0.05 |
| Aortic τ vs Temporal τ | 0.99 | 0.08 |

As demonstrated below, this finding creates promising opportunities in cardiovascular monitoring by suggesting that aortic t can be accurately replaced by carotid or temporal t. Because carotid and temporal pressure waveforms both can be measured significantly more easily (e.g. using a tonometer) in comparison to the aortic pressure waveform (acquired by invasive means), the foregoing finding provides a way to estimate CO noninvasively.

In particular, as described in the above-mentioned Westerhof article, as well as G. de Simone, M. J. Roman, M. J. Koren, G. A. Mensah, A. Ganau, and R. B. Devereux, “Stroke Volume/Pulse Pressure Ratio and Cardiovascular Risk in Arterial Hypertension,” Hypertension, vol. 33, no. 3, pp. 800-805, March 1999, doi: 10.1161/01.HYP.33.3.800 and D. Chemla et al., “Total arterial compliance estimated by stroke volume-to-aortic pulse pressure ratio in humans,” American Journal of Physiology-Heart and Circulatory Physiology, vol. 274, no. 2, pp. H500-H505, February 1998, doi: 10.1152/ajpheart.1998.274.2.H500, there has been a commonly used formula in clinical practice that suggests total arterial compliance, C, is proportional to stroke volume, SV, divided by aortic pulse pressure, aPP:

$$C ∼ SV / aPP or C = k \times SV / aPP \qquad ( Equation 2 )$$

In addition, the following equation holds:

$$R = {aP}_{mean} / CO . \qquad ( Equation 3 )$$

where aPₘₑₐₙ is the aortic mean pressure and CO is the cardiac output, which equals heart rate, HR, multiplied by SV, namely:

$$CO = SV \times HR \qquad ( Equation 4 )$$

wherein HR equals 1/T, where T is the time duration of a heartbeat.

Starting from Equation. 2, the following computations are made:

$$C = k \times SV / aPP ⇒ RC = R \times k \times SV / aPP$$

substituting in Equation 1 provides:

$$⇒ RC = R \times k \times SV / aPP ⇒ τ = k \times R \times SV / aPP$$

substituting in Equation 3 provides:

$$⇒ τ = k \times {aP}_{mean} / CO \times SV / aPP$$

and substituting in Equation 4 and HR=60/T provides:

$$⇒ τ = k \times {aP}_{mean} / ( SV \times HR ) \times SV / aPP ⇒ τ = k \times T / 60 \times {aP}_{mean} / aPP \qquad ( Equation 5 )$$

$$or$$

$$τ = {k}^{′} \times T \times {aP}_{mean} / aPP$$

where k′=k/60.

The constant k′ may be defined by fitting the true t and the right hand side of Equation 5 using the synthetic data generated using the calibrated one-dimensional arterial tree model described above. Using a linear fitting, f(x)=k′×x of the synthetic data for true τ against the product T×aPₘₑₐₙ/aPP of Equation 5, yields the relationship:

$$τ = 0.797 \times T \times {aP}_{mean} / aPP \qquad ( Equation 6 )$$

where k′=0.797 (0.794, 0.8 with 95% confidence bounds) and a goodness of fit sum of squares error, R²=0.94. FIG. 3 is a scatterplot showing correlation between t and the product of the heart beat duration times the mean arterial pressure divided by the aortic pulse pressure according to Equation 6.

Equation 6 provides a simple and fast formula to compute t using T, aPₘₑₐₙ, and aPP, without requiring the entire aortic pressure waveform.

Next, correlation of the theoretical τ formula computed with Equation 6 was validated for clinical data from an in vivo population. Following the same fitting process that was performed for the synthetic data, the age-dependence of the k′ coefficient was evaluated. Initially, it was hypothesized that k′ varies with age and accordingly the in vivo population was divided in three age groups: 30-39 years, 40-49 years, and >50 years. A fitting analysis was conducted for each age group for the linear fit models, yielding the results set forth in Table 2. The results of the analysis using the in silico data are presented to facilitate comparison.

TABLE 2

| | Coefficients | |
|---|---|---|
| | (with 95% confidence bounds) | R² |
| Linear fitting | k′ = 0.797 (0.794, 0.8) | 0.94 |
| (in silico) | | |
| Linear fitting | k′ = 0.664 (0.661, 0.666) | 0.86 |
| (all ages) | | |
| Linear fitting | k′ = 0.667(0.662, 0.672) | 0.86 |
| (30-39 years) | | |
| Linear fitting | k′ = 0.668 (0.665, 0.671) | 0.89 |
| (40-49 years) | | |
| Linear fitting | k′ = 0.654 (0.648, 0.659) | 0.82 |
| (>50 years) | | |

As shown by the results in Table 2, the value of k′ does not vary significantly across the age groups; thus suggesting that the coefficients may be considered age-independent.

### Proposed Clinical Applications for Non-Invasive Cardiac Output Estimation

Cardiac output (CO) is a primary determinant of global oxygen transport from the heart to the human body. It is widely considered to be a powerful index for predicting clinical outcomes and effectively assessing cardiovascular disease, for example, as described in S. Jhanji, J. Dawson, and R. M. Pearse, “Cardiac output monitoring: basic science and clinical application,” Anaesthesia, vol. 63, no. 2, pp. 172-181, 2008, doi: 10.1111/j.1365-2044.2007.05318.x and D. LeDoux, M. Astiz, C. Carpati, and E. Rackow, “Effects of perfusion pressure on tissue perfusion in septic shock,” Critical Care Medicine, vol. 28, no. 8, pp. 2729-2732 August 2000. Critically ill or intensive care unit (ICU) patients often require continuous assessment of CO for diagnostic purposes or for guiding therapeutic interventions, as described in H. Berkenstadt et al., “Stroke volume variation as a predictor of fluid responsiveness in patients undergoing brain surgery,” Anesthesia & Analgesia, vol. 92, no. 4, pp. 984-989, April 2001, doi: 10.1097/00000539-200104000-00034, M. McKendry, H. McGloin, D. Saberi, L. Caudwell, A. R. Brady, and M. Singer, “Randomised controlled trial assessing the impact of a nurse delivered, flow monitored protocol for optimisation of circulatory status after cardiac surgery,” BMJ, vol. 329, no. 7460, p. 258, July 2004, doi: 10.1136/bmj.38156.767118.7C and N. Lees, M. Hamilton, and A. Rhodes, “Clinical review: goal-directed therapy in high risk surgical patients,” Crit Care, vol. 13, no. 5, p. 231, 2009, doi: 10.1186/cc8039. Despite the diagnostic importance of CO, the convenience of its measurement is significantly limited due to potential cost, the need for special equipment and training. Moreover, the state-of-the-art of methods for obtaining CO suffer from practical limitations as described in L. S. Nguyen and P. Squara, “Non-Invasive Monitoring of Cardiac Output in Critical Care Medicine,” Front Med (Lausanne), vol. 4, November 2017, doi: 10.3389/fmed.2017.00200, such that the majority of the previously known techniques appear better suited for trend-monitoring rather than measuring absolute CO.

In contrast to CO measurements, peripheral measurements of such physiologic inputs as systolic and diastolic brachial pressure or carotid and radial pressure waveforms are fully noninvasive and may be easily monitored by a clinician on a regular basis, as described in P. L. Marino, The ICU book, 2. ed. Baltimore: Lippincott Williams & Wilkins, 1998. Accordingly, there is substantial motivation to develop noninvasive methods for estimating central cardiovascular quantities (e.g., central systolic blood pressure). Despite the broad range of studies exerted to acquire central pressure estimates, heretofore no methods to precisely predict absolute CO using peripheral measurements have been discovered. The insights and inventions set forth in this disclosure change that paradigm, presenting novel methods for estimating CO that leverage the capacity of machine learning.

Described below are simple, fast, convenient and cost-efficient methods to estimate real time CO that employ as an input a small number of non-invasive easily obtained clinical data, specifically, brachial systolic and diastolic blood pressure, heart rate (HR), and/or pulse wave velocity (PWV) data. PWV may be routinely measured in clinical practice with satisfactory repeatability, and has been identified as an independent predictor of clinical outcomes, as described in S. Laurent et al., “Expert consensus document on arterial stiffness: methodological issues and clinical applications,” European Heart Journal, vol. 27, no. 21, pp. 2588-265 September 2006, doi: 10.1093/eurheartj/eh1254, thus making PWV a valuable adjunct to blood pressure (BP) measurements in routine assessments of risk. Two proposed methods are described below that use the same set of required (input) measurements, namely, the cuff SBP and DBP, the uncalibrated carotid, femoral, and radial pressure waveforms, and HR, which are non-invasively determined and readily available.

In a first method, starting from Eq. 2, the following computations may be performed:

$$C = k \times SV / aPP ⇒ SV = 1 / k \times C \times aPP ⇒ SV = {k}^{″} \times C \times aPP$$

substituting in Equation 4 and HR=60/T:

$$⇒ CO = {k}^{′′′} \times C \times aPP \times 1 / T \qquad ( Equation 7 )$$

Equation 7 provides computation of an estimate for CO provided that total arterial compliance, C, aortic pulse pressure, aPP, and, heart cycle, T, are known.

The above-mentioned WO application, as summarized in V. Bikia, G. Rovas, S. Pagoulatou, and N. Stergiopulos, “Determination of Aortic Characteristic Impedance and Total Arterial Compliance From Regional Pulse Wave Velocities Using Machine Learning: An in-silico Study,” Front. Bioeng. Biotechnol., vol. 9, p. 649866, May 2021, doi: 10.3389/fbioe.2021.649866, describes a method for predicting C from cuff BP measurements, specifically, systolic (SBP) and diastolic blood pressure (DBP) and arterial stiffness, as determined from carotid-femoral pulse wave velocity (cfPWV) and/or carotid-radial pulse wave velocity (crPWV). In addition, as discussed below, aPP can be estimated from cuff SBP and DBP using machine learning. Finally, T is computed as 60/HR.

The carotid-femoral pulse wave velocity (cfPWV) and carotid-radial pulse wave velocity (crPWV) may be calculated employing a foot-to-foot algorithm using the tangential method described in O. Vardoulis, T. G. Papaioannou, and N. Stergiopulos, “Validation of a novel and existing algorithms for the estimation of pulse transit time: advancing the accuracy in pulse wave velocity measurement,” American Journal of Physiology-Heart and Circulatory Physiology, vol. 304, no. 11, pp. H1558-H1567, June 2013, doi: 10.1152/ajpheart.00963.2012. Pulse transit times are computed between the two arterial sites, the left carotid and left femoral artery, and the left carotid and the left radial artery, respectively. The tangential method uses the intersection of two tangents on the arterial pressure wave, that is, the tangent passing through the systolic upstroke and the horizontal line passing through the minimum of the pressure wave. The travel lengths may be determined by summing the lengths of the arterial segments within the transmission paths. The value of each PWV then may be calculated by dividing the total travel length by the pulse transit time.

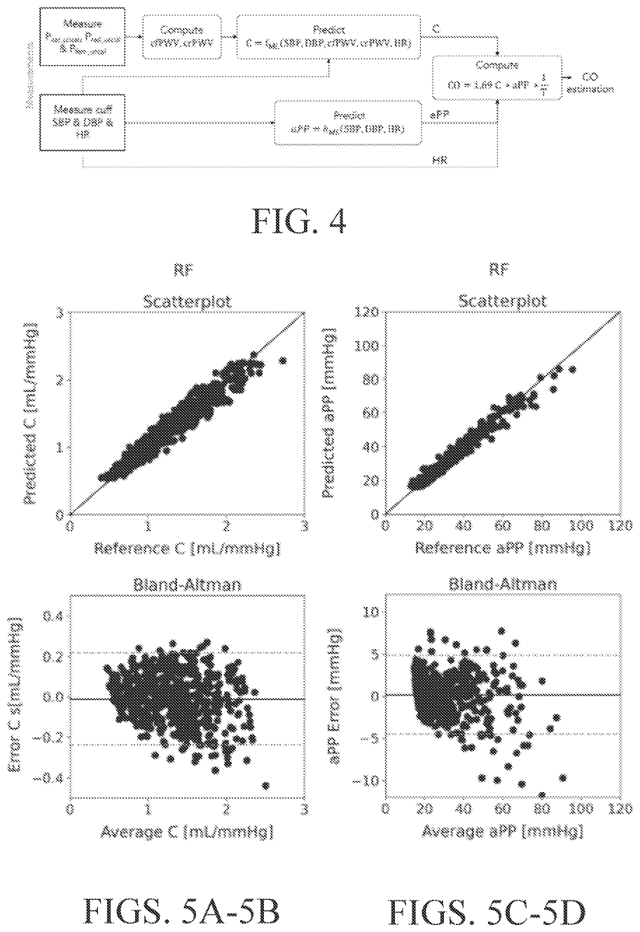

The required patient physiologic input measurements include: the uncalibrated carotid, radial, and femoral pressure waves, the cuff (brachial) SBP and DBP, and HR. FIG. 4 provides a schematic representation of the method, in which the noninvasively measured patient values are input into software that computes the pressure wave velocities and then employs a machine learning approach to analyze the synthetic data from the calibrated one-dimensional arterial tree to generate predictions for total arterial compliance C and aortic pulse pressure aPP. Those predicted values then are used, together with the measured heart rate, HR, to compute CO using Equation 7.

The foregoing approach was validated using the synthetic data generated from the one-dimensional arterial tree model. In particular, the data was divided into three sets: 60% of the data was used as a training set, 20% of the data was used as a validation set, and the remaining 20% of the data was used as a test set. The training set was to estimate the coefficient k′″ in Equation 7 and also to train the machine learning models to predict total arterial compliance (C) and aortic pulse pressure (aPP). The validation set was used for hyperparameter tuning the machine learning models, while the test set was used to assess the performance of the trained models.

When fit to the training set, the coefficient k′″ in Equation 7 was determined to be 1.69 (with an R2=0.81). C was estimated from systolic BP (SBP) and diastolic BP (DBP), HR and cfPWV via regression analysis (accuracy: RMSE=0.12 mL/mmHg, r=0.96). Estimations of aPP were obtained using as inputs SBP, DBP, and HR via regression analysis (accuracy: RMSE=2.4 mmHg, r=0.99). FIGS. 5A and 5B are scatter plots showing the true and the estimated values for C. FIGS. 5C and 5D are scatter plots showing the true and the estimated values of aortic pressure pulse, aPP.

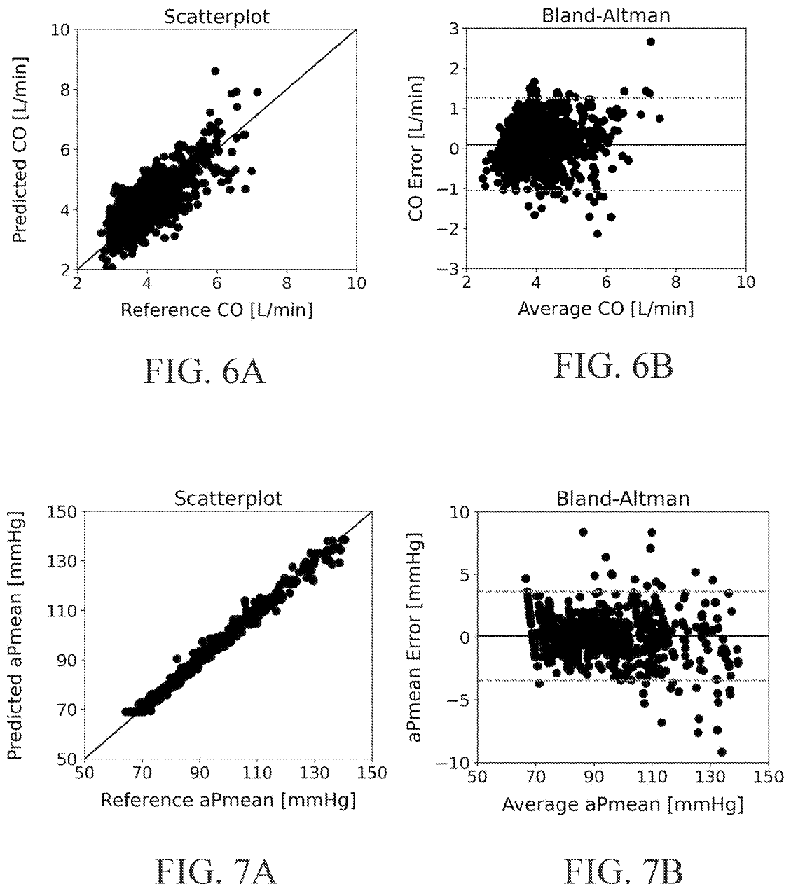

Next, using the estimated coefficient k′″, C, and aPP, cardiac output CO was computed using Equation 7. As depicted in FIGS. 6A and 6B, estimated CO was found to be in agreement with true CO values (accuracy: RMSE=0.6 L/min, r=0.8).

Adopting a slightly different approach, an alternative method is formulated in which CO may be based on the formula:

$$CO = {aP}_{mean} / R \qquad ( Equation 8 )$$

where aPₘₑₐₙ is the aortic mean pressure, and R is the peripheral resistance computed using τ as R=τ/C. As demonstrated above, the latter equation holds for the aortic τ. However, based on the above-mentioned discovery that aortic τ and carotid τ are the same, the value of τ may be calculated from the carotid pressure waveform. In particular, the uncalibrated carotid pressure waveform may be calibrated based on the well-established assumptions that mean arterial pressure, MAP, may be computed as (SBP+2DBP)/3) and DBP remain constant across all major arteries (including the carotid artery and the brachial artery). C may be predicted from SBP, DBP, cfPWV, crPWV and HR using regression analysis, as described for the preceding method. Finally, aPₘₑₐₙ may be estimated from SBP, DBP, and HR using regression analysis. Estimates of aPₘₑₐₙ may be obtained using as inputs SBP, DBP, and HR via regression analysis (RMSE=1.81 mmHg, r=0.99). FIGS. 7A and 7B are scatter plots comparing predicted aortic mean pressure to true aortic mean pressure.

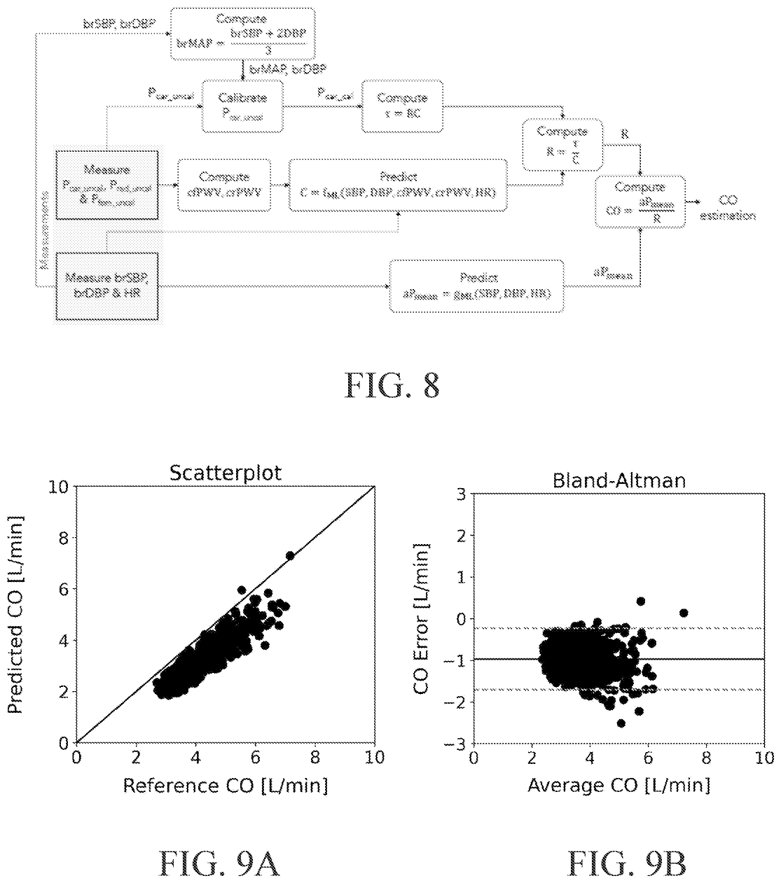

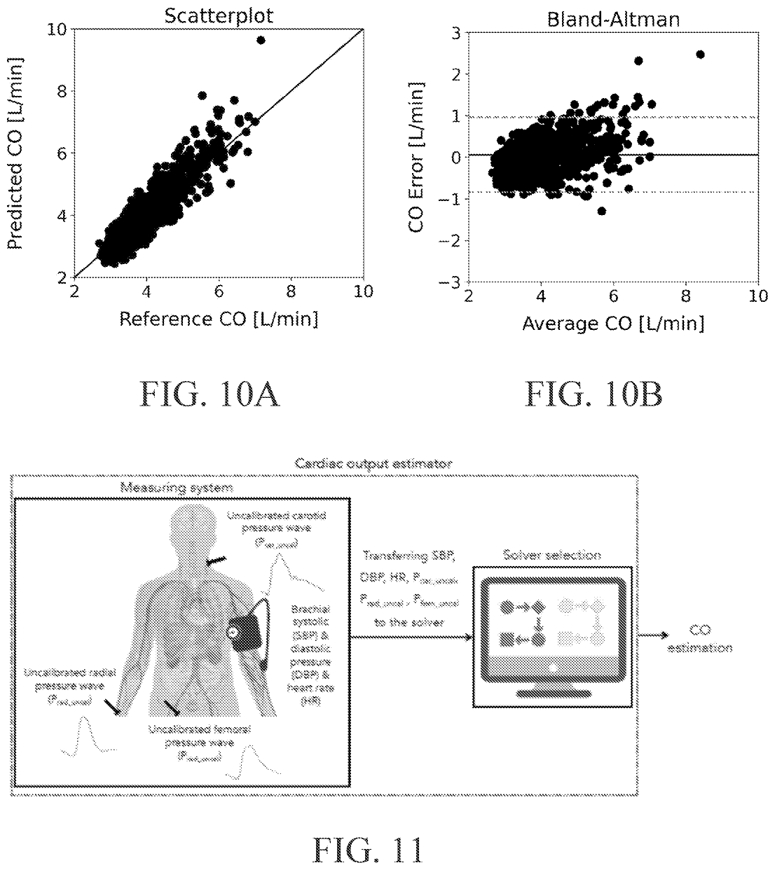

FIG. 8 is a schematic representation of a method of using the above parameters and Equation 8 to estimate CO. FIGS. 9A and 9B depict preliminary results obtained using this second method of FIG. 8 to estimate CO, and show correlation between with the estimated CO and true CO values (accuracy: RMSE=1.04 L/min, r=0.9). However, there is a consistent underestimation of the estimated CO compared to the true CO values (bias=−0.97 L/min). The percentage of underestimation was found to be constant across all CO values and equal to −32%. As a result, in order to correct for the underestimation, the estimated CO was then multiplied by a factor 1.32. The value of the corrective factor remains to be evaluated in a human population. FIGS. 10A and 10B depict preliminary results obtained by applying the correction factor to the second method of FIG. 8 to estimate CO, and show agreement between with the estimated CO and true CO values (accuracy: RMSE=0.46 L/min, r=0.9).

Referring now to FIG. 11, a patient monitoring system programmed with software that embodies either or both of the methods of estimating CO described for the above. The monitoring system preferably collects patient physiologic values using three sensors (e.g., pen-like tonometers, optical sensors, accelerometers or similar devices) to collect uncalibrated carotid, femoral, and radial pressure waveforms, and a cuff-based device (e.g. sphygmomanometer, oscillometer or other similar device) that measures brachial SBP and DBP values and HR. HR alternatively may be obtained from any of multiple wearable devices (e.g. smart watch or fitness bands). These values are transmitted to a console programmed with solver software for estimating CO, as described above.

Hardware for implementing the inventive system may be as described for FIG. 2 of the above-incorporated International Application Publication WO 2021/033097. The console of FIG. 11 may comprise a computer, e.g., laptop, desktop, or tablet that is programmed with the estimator software as described herein, and preferably includes at least one processor, memory, non-volatile storage, transceiver, power source, and one or more input devices and output devices. The processor may be a conventional multi-core processor, such as an Intel CORE i5 or i7 processor, while the memory may comprise volatile (e.g. random-access memory (RAM)), non-volatile (e.g. read-only memory (ROM)), flash memory, or any combination thereof. A transceiver may be provided to receive and/or transmit information to and from other components in the monitoring system, including the sensors and cuff-based device, using any well-known communication infrastructure facilitating communication over wired or wireless connection, such as any IEEE 802 standard.

The console containing the solver preferably includes a power source that connects to a standard wall outlet and/or may include a battery. Nonvolatile storage preferably is provided that may include removable and/or non-removable storage, such as, solid state disk memory of magnetic hard drive. One or more input devices coupled to, or integrated into, console for inputting data, and may include, for example, a keyboard or touchscreen, a mouse and/or a pen. The one or more input devices may be used to input patient specific information into the estimator, e.g., height, age, weight, gender, and identity, and/or to modify the sampling rate of sensors or the frequency with which pressure measurements are taken with cuff-based device. One or more output devices may be coupled to or integrated into console for outputting or otherwise displaying data, such as video screen, printer or plotter. An output device further may include a speaker or alarm bell that may be activated if a monitored estimated parameter, such as CO, falls below a clinically significant threshold indicating patient distress.

In one embodiment, the operating system for the console and the solver software may be stored in non-volatile storage, and may comprise, e.g., Microsoft Windows or Linux, as well as the necessary drivers for the input and output devices.

As described above, the solver CO of the present disclosure can accurately estimate from simple formulas, using non-invasive measurement of patient physiologic data, real time values of estimated CO. Preliminary validation of the described methods demonstrates a cost-effective and readily available technique for non-invasively monitoring CO in a clinical setting.

While various illustrative embodiments of the invention are described above, it will be apparent to one skilled in the art that various changes and modifications may be made therein without departing from the invention.

What is claimed is:

1. A system for non-invasively estimating cardiac output of a patient, the system comprising:
a plurality of sensors configured to be applied to the patient, the plurality of sensors providing as outputs at least a carotid artery pressure wave and at least one of a radial artery pressure wave or a femoral artery pressure wave;
a cuff configured to be applied to the patient to generate values of systolic and diastolic blood pressures and heart rate;
a console including a processor and non-volatile storage, wherein the non-volatile storage stores instructions for a solver that, when executed by the processor:
receives the outputs of the plurality of sensors and the values of systolic and diastolic blood pressures and heart rate generated by the cuff;
computes at least one of a carotid artery to femoral artery pulse wave velocity and a carotid artery to radial artery pulse wave velocity using the outputs of the plurality of sensors;
computes an estimated value of aortic pulse pressure using the values of systolic and diastolic blood pressures and heart rate generated by the cuff;
computes an estimated value of total arterial compliance using the values of systolic and diastolic blood pressures and heart rate generated by the cuff and at least one of a carotid artery to femoral artery pulse wave velocity and a carotid artery to radial artery pulse wave velocity; and
computes and outputs for display an estimated value of cardiac output based on the estimated value of total arterial compliance, the estimated value of aortic pulse pressure and the heart rate.

2. The system of claim 1, wherein computing an estimated value of aortic pulse pressure comprises analyzing a database of synthetic data generated from a calibrated one-dimensional arterial tree model.

3. The system of claim 2, wherein analyzing the database of synthetic data includes employing a machine learning algorithm.

4. The system of claim 1, wherein computing an estimated value of total arterial compliance comprises analyzing a database of synthetic data generated from a calibrated one-dimensional arterial tree model.

5. The system of claim 4, wherein analyzing the database of synthetic data includes employing a machine learning algorithm.

6. The system of claim 1, wherein a first one of the plurality of sensors is disposed on a patch configured to be disposed on skin of the patient in a vicinity of a proximal arterial site and a second one of the plurality of sensors is disposed on a patch configured to be disposed on skin of the patient in a vicinity of a distal arterial site.

7. A method for estimating cardiac output of a patient using non-invasively measurable physiologic data, the method comprising:
receiving as outputs from a plurality of sensors at least a carotid artery pressure wave and at least one of a radial artery pressure wave or a femoral artery pressure wave;
receiving from values of systolic and diastolic blood pressures;
receiving a heart rate for the patient;
computing at least one of a carotid artery to femoral artery pulse wave velocity and a carotid artery to radial artery pulse wave velocity using the outputs from the plurality of sensors;
computing an estimated value of aortic pulse pressure using the values of systolic and diastolic blood pressures and heart rate;
computing an estimated value of total arterial compliance using the values of systolic and diastolic blood pressures and heart rate and at least one of a carotid artery to femoral artery pulse wave velocity and a carotid artery to radial artery pulse wave velocity; and
computing and outputting for display an estimated value of cardiac output based the estimated value of total arterial compliance, the estimated value of aortic pulse pressure and the heart rate.

8. The method of claim 7, wherein computing an estimated value of aortic pulse pressure comprises analyzing a database of synthetic data generated from a calibrated one-dimensional arterial tree model.

9. The method of claim 8, wherein analyzing the database of synthetic data includes employing a machine learning algorithm.

10. The method of claim 7, wherein computing an estimated value of total arterial compliance comprises analyzing a database of synthetic data generated from a calibrated one-dimensional arterial tree model.

11. The method of claim 10, wherein analyzing the database of synthetic data includes employing a machine learning algorithm.

12. The method of claim 1, further comprising applying a first one of the plurality of sensors on skin of the patient in a vicinity of a proximal arterial site and applying a second one of the plurality of sensors on skin of the patient in a vicinity of a distal arterial site.

13. A system for estimating cardiac output of a patient, the system comprising:
a plurality of sensors configured to be applied to the patient, the plurality of sensors providing as outputs at least a carotid artery pressure wave and at least one of a radial artery pressure wave or a femoral artery pressure wave;
a cuff configured to be applied to the patient to generate values of systolic and diastolic blood pressures and heart rate;
a console including a processor and non-volatile storage, wherein the non-volatile storage stores instructions for a solver that, when executed by the processor:
receives the outputs of the plurality of sensors and values of systolic and diastolic blood pressure and heart rate generated by the cuff;
computes a mean arterial pressure as a weighted average of the values of systolic and diastolic blood pressure generated by the cuff;
computes a time decay constant for diastolic aortic pressure based on the mean arterial pressure;
computes at least one of a carotid artery to femoral artery pulse wave velocity and a carotid artery to radial artery pulse wave velocity using the outputs of the plurality of sensors;
computes an estimated value of total arterial compliance using the outputs of the cuff and at least one of a carotid artery to femoral artery pulse wave velocity and a carotid artery to radial artery pulse wave velocity;
computes an estimated value of peripheral arterial resistance based on the time decay constant and the total arterial compliance;
computes an estimated value of aortic mean pressure using the values of systolic and diastolic blood pressure and heart rate generated by the cuff; and
computes and outputs for display an estimated value of cardiac output based on the estimated value of aortic mean pressure and estimated value of peripheral arterial resistance.

14. The system of claim 13, wherein computing an estimated value of total arterial compliance and an estimated value of aortic mean pressure comprises analyzing a database of synthetic data generated from a calibrated one-dimensional arterial tree model.

15. The system of claim 14 wherein analyzing the database of synthetic data includes employing a machine learning algorithm.

16. The system of claim 13 wherein the values of systolic and diastolic blood pressures generated by the cuff are uncalibrated.

17. The system of claim 13, wherein a first one of the plurality of sensors is disposed on a patch configured to be disposed on skin of the patient in a vicinity of a proximal arterial site and a second one of the plurality of sensors is disposed on a patch configured to be disposed on skin of the patient in a vicinity of a distal arterial site.

18. A method for estimating cardiac output of a patient using non-invasively measurable physiologic data, the method comprising:
receiving as outputs from a plurality of sensors at least a carotid artery pressure wave and at least one of a radial artery pressure wave or a femoral artery pressure wave;
receiving values of systolic and diastolic blood pressures;
receiving a heart rate of the patient;
computing a mean arterial pressure as a weighted average of the values of systolic and diastolic blood pressure;
computing a time decay constant for diastolic aortic pressure based on the mean arterial pressure;
computing at least one of a carotid artery to femoral artery pulse wave velocity and a carotid artery to radial artery pulse wave velocity using the outputs from the plurality of sensors;
computing an estimated value of total arterial compliance using the values of systolic and diastolic blood pressures and at least one of a carotid artery to femoral artery pulse wave velocity and a carotid artery to radial artery pulse wave velocity;
computing an estimated value of peripheral arterial resistance based on the time decay constant and the total arterial compliance;
computing an estimated value of aortic mean pressure using the values of systolic and diastolic blood pressure and the heart rate; and
computing and outputting for display an estimated value of cardiac output based on the estimated value of aortic mean pressure and estimated value of peripheral arterial resistance.

19. The method of claim 18, wherein computing an estimated value of total arterial compliance and an estimated value of aortic mean pressure comprises analyzing a database of synthetic data generated from a calibrated one-dimensional arterial tree model.

20. The method of claim 19, wherein analyzing the database of synthetic data includes employing a machine learning algorithm.

21. The method of claim 18, further comprising applying a first one of the plurality of sensors on skin of the patient in a vicinity of a proximal arterial site and applying a second one of the plurality of sensors on skin of the patient in a vicinity of a distal arterial site.
